(19) United States
(12) Patent Application Publication
(10) Pub. No.: US 2006/0052692 A1
(43) Pub. Date: Mar. 9, 2006

(54) Method and arrangement for determining the position of an object in an mr device

(76) Inventor: Steffen Weiss, Hamburg (DE)

Correspondence Address: PHILIPS INTELLECTUAL PROPERTY & STANDARDS, 595 MINER ROAD, CLEVELAND, OH, 44143, US

(21) Appl. No.: 10/538,428

(22) PCT Filed: Dec. 2, 2003
(86) PCT No.: PCT/IB03/05603

§ 371 Date: Jun. 10, 2005

(30) Foreign Application Priority Data
Dec. 13, 2002 (EP) 02102755.2

(51) Int. Cl. A61B 5/05

(52) U.S. Cl. 600/424

(57) ABSTRACT

The invention relates to a method of determining the position of an object, such as for example a medical intervention instrument, located in the examination area of an MR device. For this purpose, a high-frequency magnetic field is generated in the examination area, which high-frequency magnetic field runs essentially parallel to a main magnetic field that is active at the same time. In the process, a component of the high-frequency magnetic field that is perpendicular to the main magnetic field is produced in the vicinity of conversion means fitted on the object, on account of which perpendicular component of the high-frequency magnetic field a nuclear resonance signal is excited. In conjunction with a gradient field, the nuclear resonance signal is detected and evaluated, so that the position of the object can be determined. Furthermore, the invention relates to an MR device and to specific components for carrying out the method.

The invention relates to a method of determining the position of an object, such as for example a medical intervention instrument, located in the examination area of an MR device. Furthermore, the invention relates to an MR device and to specific components for carrying out the method.

An apparatus of the type mentioned in the introduction is known from patent DE19844762. Therein, it is stated that a nuclear resonance signal from a limited region of an examination area that is to be mapped can be considerably increased if a separate coil arrangement that is connected to a resonant circuit is positioned in the direct vicinity of the region. By means of this arrangement, an additional signal is generated which allows conclusions to be drawn about the position of the coil arrangement. In order that the coil arrangement, which is fitted for example to a catheter, is not dependent on its alignment with respect to the main magnetic field of the MR device, it is proposed to construct the coil arrangement from three decoupled coil arrangements, with the coil axes in each case being perpendicular to one another. The signal generated by such a coil arrangement is merely contained in the other nuclear resonance signals as an additional signal, as a result of which it is often difficult to distinguish from said nuclear resonance signals. As a result, the detectability or determination of the position of the coil arrangement is highly impaired.

It is therefore an object of the present invention to specify a method and the means for carrying out the method, by means of which better detectability and/or determination of the position of an object in an MR device is possible.

The object is achieved by a method for determining the position of an object located in the examination area of an MR device, having the steps
a) generation of a high-frequency magnetic field in the examination area, which high-frequency magnetic field runs essentially parallel to a main magnetic field that is active at the same time, with a component of the high-frequency magnetic field that is perpendicular to the main magnetic field being generated from the high-frequency magnetic field by inversion means fitted on the object, in the vicinity thereof,
b) detection of the nuclear resonance signal excited as a result of the perpendicular component of the high-frequency magnetic field, in conjunction with a gradient magnetic field,
c) evaluation of the nuclear resonance signals and determination of the position of the object.

To achieve the object, instead of the high-frequency magnetic fields known to date which run perpendicular to the main magnetic field, a new high-frequency magnetic field is generated which in the examination area of the MR device runs essentially parallel to the main field that is active at the same time. On account of the parallel running, initially no nuclear resonance signal is excited. Furthermore, in the examination area there are conversion means which in the vicinity thereof generate, from the high-frequency magnetic field, a component of the high-frequency magnetic field that is perpendicular to the main magnetic field. These perpendicular components effect excitement of a nuclear resonance signal in the vicinity of the conversion means. By contrast with conventional imaging, where a nuclear resonance signal is excited in a large area of the subject under examination, in this case there is only excitement of a nuclear resonance signal in the vicinity of the conversion means. Analogously to the conventional imaging method, this nuclear resonance signal is detected in conjunction with at least one gradient magnetic field using the known detection means. By superposing the main magnetic field with the gradient magnetic field, a local resolution of the detected signal is obtained along the gradient, from which, following evaluation of the signals, in the known way the position of the conversion means and thus of the object to which the conversion means are fitted can be determined.

Using this method, it is possible to carry out determination of the position of an object independently of the actual imaging. In particular, compared to the method disclosed in DE19844762, in which the signals of the microcoils exist as a small signal increase in addition to the other nuclear resonance signals, using the method according to the invention only signals from the areas in the vicinity of the conversion means are obtained, which makes evaluation considerably easier and more accurate. If the user is only interested in the position of the object in one dimension, then the decoupling of the actual imaging from the determination of the position allows a considerably faster reception and evaluation process, since for example during the reception of the signals only one gradient field needs to be active and the signal need only be evaluated in respect of one dimension. However, it is also conceivable for a number of gradient fields to be used, in order for example to determine the position in three dimensions. On account of the decoupling of the imaging from the determination of the position, it is possible in the imaging to use a local resolution other than the determination of the position, or to determine the position from only a single projection per spatial direction.

The object is also achieved by means of an MR device for carrying out a method as mentioned above, having
a) means for generating a main magnetic field in an examination area,
b) means for generating a high-frequency magnetic field in the examination area, which high-frequency magnetic field runs essentially parallel to the main magnetic field,
c) means for generating at least one gradient magnetic field,
d) means for detecting nuclear resonance signals,
e) an evaluation unit for evaluating the nuclear resonance signals,
f) a control unit for controlling the aforesaid components such that the following steps are carried out:
f1) generation of a high-frequency magnetic field in the examination area, which high-frequency magnetic field runs essentially parallel to a main magnetic field that is active at the same time, with a component of the magnetic high-frequency field that is perpendicular thereto being generated by conversion means fitted on the object, in the vicinity thereof,
f2) detection of the nuclear resonance signal excited as a result of the perpendicular component of the high-frequency magnetic field, in conjunction with a gradient magnetic field,
f3) evaluation of the nuclear resonance signals and determination of the position of the object.

The main magnetic field of the MR device that is required to flow through the examination area can in a known manner be formed for example with an air-core coil, the windings of which are designed to be superconductive depending on the desired field strength or with permanent magnets. The high-frequency magnetic field that is parallel to the main magnetic field can be generated, for example, by the coil arrangement of the main magnetic field additionally being acted upon by an electrical pulse, as a result of which additionally a high-frequency magnetic field parallel to the main magnetic field is produced in the determination area. In particular, in the case of a superconductive configuration of the coil of the main magnetic field, the use of a separate coil arrangement is also possible as an alternative, as claimed in claim 3, in order to generate the desired high-frequency magnetic field. Such a coil arrangement can for example be wound in a manner parallel to or alternating with the windings of the coil arrangement of the main magnetic field on a common carrier. It is also possible to design an independent component for an MR device, on which component the coil arrangement is arranged and which component is positioned inside or outside the coil arrangement of the main magnetic field. Such a component can then be marketed, for example, as an optional additional component for an MR device.

The nuclear resonance signal excited by the high-frequency magnetic field is detected using known detection means, such as antennas or coils. These detection means can be used both for detecting nuclear resonance signals for position determination and for detecting imaging nuclear resonance signals (to generate imaging nuclear resonance signals, the MR device then also additionally contains means for generating a high-frequency magnetic field in the examination area, which high-frequency magnetic field runs essentially perpendicular to the main magnetic field). The detected nuclear resonance signal passes into an evaluation unit which, in addition to a possible image reconstruction, also evaluates the nuclear resonance signal for position determination purposes. Furthermore, the MR device contains a control unit which controls the other aforesaid components in a manner such that a method according to the invention can be carried out. Both the evaluation unit and the control unit can be configured such that they can be programmed, and this enables them, as claimed in claim 10, to carry out their respective task.

The conversion means, which are necessary in order to carry out the method according to the invention, may be formed for example by an active unit having a transmitter and a receiver, wherein the high-frequency magnetic field is received by a receiving antenna and is emitted again, in a manner rotated through 90° in spatial terms, by an emitting antenna. One possible alternative is, as claimed in claim 4, the configuration using a coil arrangement, which coil arrangement is dimensioned such that it can be fitted to the object whose position is to be determined. The coil of the coil arrangement must be positioned such that its coil axis is at an angle other than 90° with respect to the high-frequency magnetic field. Then a current is induced in the coil arrangement, by means of which current a high-frequency magnetic field is produced in the vicinity of the coil arrangement, which high-frequency magnetic field contains a component that is perpendicular to the high-frequency magnetic field that is being excited. The perpendicular component is at its greatest when the coil axis of the coil forms an angle of 45° with respect to the high-frequency magnetic field. As claimed in claim 5, it can be additionally increased, with the resonant frequency preferably corresponding to the frequency of the high-frequency magnetic field. If the object moves during examination of the subject, then the geometric condition cannot under some circumstances always be achieved. Therefore, as claimed in claim 6, a coil arrangement having a number of coils is proposed, where the coil axes of the coils should not assume an angle of 90° with respect to one another since then the high-frequency magnetic fields of the individual coils, which high-frequency magnetic fields are perpendicular to the main magnetic field, would eliminate each other out.

If the MR device is used for examinations or operations using a medical intervention instrument, then as claimed in claim 7 such a coil arrangement can be fitted to the instrument, in order to determine its position, in particular the position of the tip of the instrument, during the examination and to display this position to the user. When using a catheter as claimed in claim 8, the conversion means can be particularly easily fitted to the catheter by means of a carrier body as claimed in claim 9.

The invention will be further described with reference to examples of embodiments shown in the drawings to which, however, the invention is not restricted.

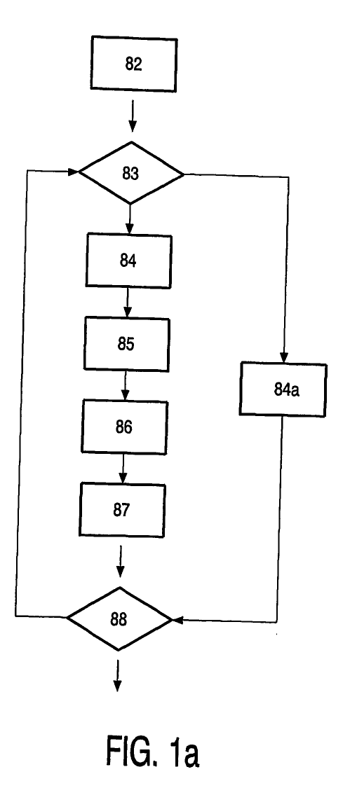

FIGS. 1a and 1b in each case show a flowchart of a method according to the invention,

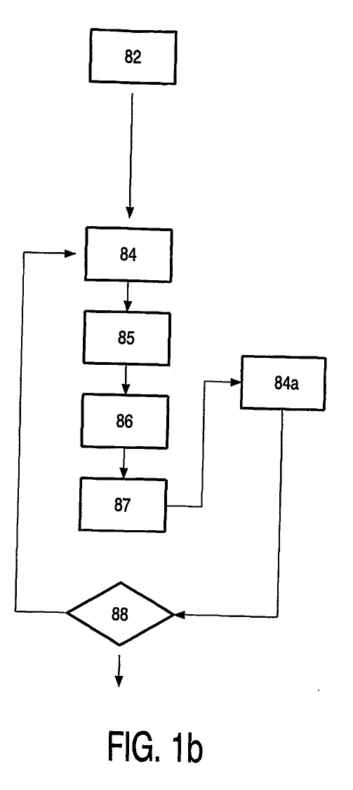

FIG. 2 shows an MR device,

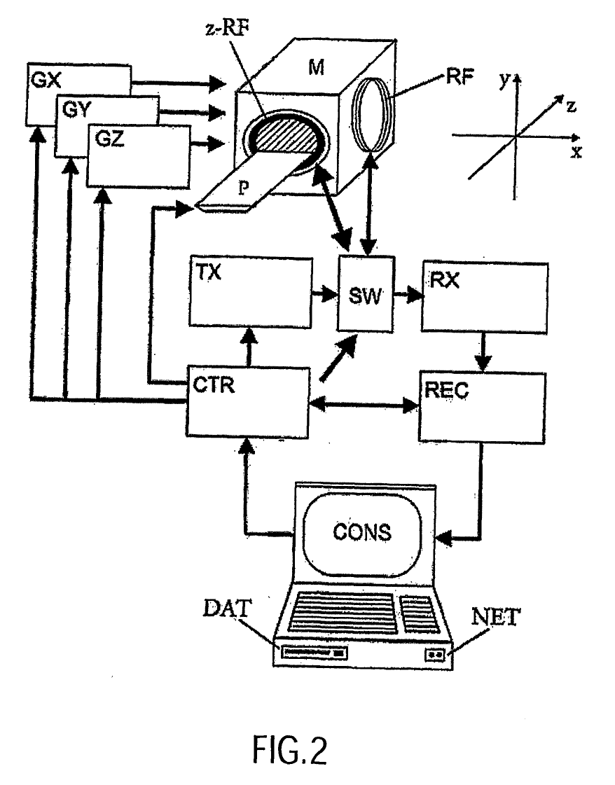

FIG. 3 shows the arrangement, in principle, of possible conversion means,

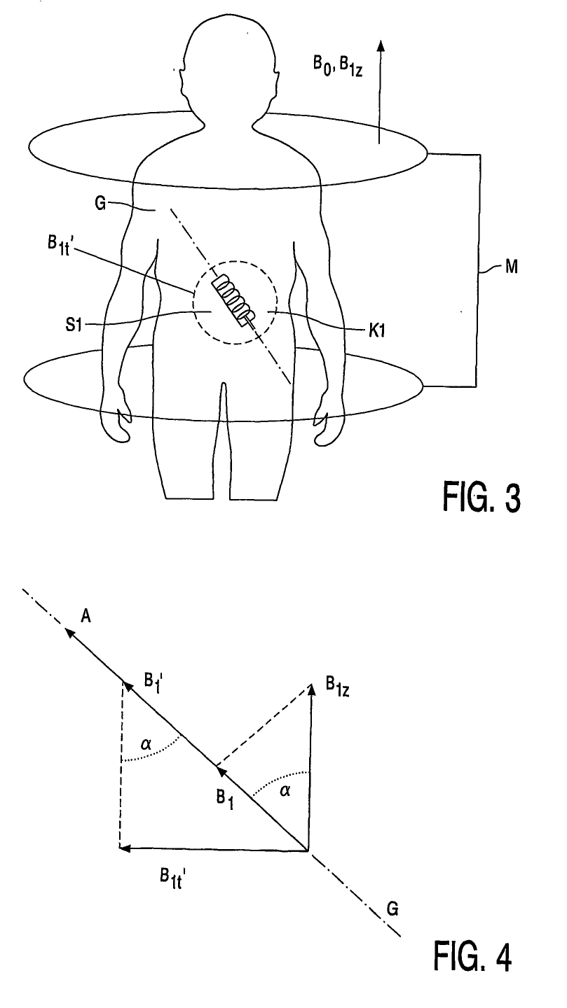

FIG. 4 shows a vectorial view of the magnetic fields in the vicinity of the conversion means,

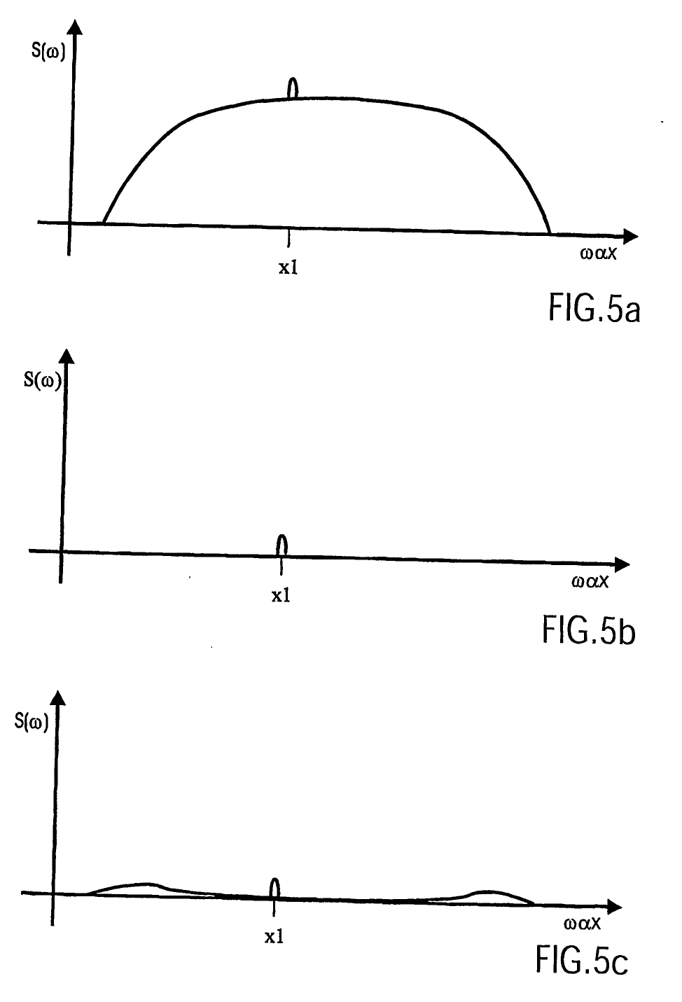

FIGS. 5a to 5c show detected signals in one dimension, and

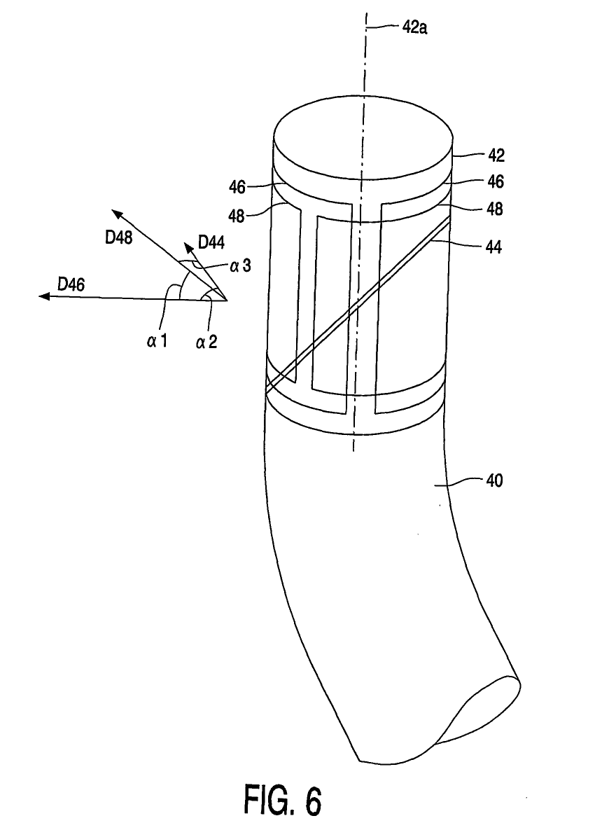

FIG. 6 shows the tip of a catheter having conversion means.

FIG. 1a shows a method according to the invention in the form of a flowchart. In step 82, the main magnetic field of an MR device is activated. Such an MR device is shown schematically in FIG. 2. In the center of the apparatus there is a main magnet M, which in the examination area generates an essentially homogeneous, steady-state main magnetic field having a flux density of, for example, 1.5 or 3 Tesla in the z-direction. The magnet M is conventionally a superconductive electromagnet, so that step 82, on account of the duration of activation, is generally carried out not before each examination but rather once at the start of a period of operation. A patient table P, on which a patient or other subject under examination is located during an examination, can be brought toward the magnet M. The field direction of the main magnetic field typically runs parallel to the longitudinal direction of the patient table P. Furthermore, an arrangement of gradient coils GX, GY, GZ is provided, which gradient coils are supplied with power by means of gradient amplifiers (not shown in greater detail). Therefore, the gradient pulses in any desired spatial directions, which are required for the various pulse sequences, can be generated in the examination area. A first high-frequency coil arrangement RF is used on the one hand to radiate, in a known manner, high-frequency magnetic pulses, the magnetic field direction of which runs approximately perpendicular to the direction of the main magnetic field, into the examination area, and on the other hand to receive MR signals from the examination area. A second high-frequency coil arrangement z-RF is used to flow through the examination area of the MR device with a high-frequency magnetic field that runs essentially parallel to the main magnetic field. On account of this parallel running, the geometries of the high-frequency coil arrangement z-RF and of the magnet M resemble one another, for example the respective coil axes in the z-direction run along the same line. The high-frequency coil arrangement z-RF can therefore be wound onto a thin tubular carrier (not shown here) which is inserted into the magnet M and reduces the size of the examination area only to an insignificant extent.

For reciprocal operation of all coil arrangements, a switch SW that is controlled by means of the control unit CTR is provided, which switch SW switches the coil arrangement RF back and forth between a high-frequency output transmitter TX and a receiver RX. Alternatively, the coil arrangement z-RF can be coupled to the transmitter TX. However, it is also possible to use a multichannel high-frequency output transmitter TX, with one channel being used for each high-frequency coil arrangement. The transmitter TX is likewise actuated by means of the control unit CTR, which control unit CTR generates the pulse sequences required for actuation of the coil arrangements RF and z-RF and also controls the gradient coils GX, GY and GZ. In addition, the position of the patient table P is changed by means of the control unit CTR. A reconstruction unit REC digitizes and stores the MR signals transmitted by the receiver RX and then reconstructs therefrom, using known methods, subject functions of the examination area. Alternatively, the reconstruction unit REC can determine, from the transmitted signals, the position of an object located in the examination area. The reconstruction unit REC is connected to an operating console CONS, which has a monitor on which image data of the reconstructed subject functions and/or the position of an object located in the examination area are displayed. At the same time, the console CONS is used to operate the entire apparatus and to initiate the desired pulse sequences. For this purpose, the console CONS is also connected to the control unit CTR. The method according to the invention is implemented by appropriate programming of the reconstruction unit REC and of the control unit CTR. The computer programs required for this purpose can be loaded into the console CONS via a reading unit DAT with the aid of a computer program product such as a diskette or CD-ROM. Furthermore, the console CONS can be connected via a network connection NET to a data network, for example the Internet, so that the computer programs are loaded from the data network.

In step 83 of FIG. 1, a decision is made as to whether subsequently a determination of the position of an object located in the examination area is to be carried out or an image is to be generated. In the latter case, step 84a is carried out, in which in a known manner nuclear resonance signals are excited by actuation of the coil arrangement RF and, under the influence of the gradient fields GX, GY and/or GZ, are in turn received by the coil arrangement RF, evaluated by the reconstruction unit REC, and processed to produce an image that can be displayed using the console CONS. To determine the position of the object, in step 84 a high-frequency magnetic field is generated using the coil arrangement z-RF, which high-frequency magnetic field runs essentially parallel to the main magnetic field. On account of the parallelism of the magnetic fields, no nuclear resonance signal is excited in the subject under examination.

As shown in FIG. 3, a coil arrangement which from a coil S1 and a capacitance K1 forms a resonant circuit with the quality Q is located at the object. The resonant frequency of the resonant circuit corresponds approximately to the frequency of the high-frequency magnetic field B_{1z}. Assuming that the coil axis G of the coil S1 does not correspond to the direction of the high-frequency magnetic field B_{1z} and also does not run perpendicular thereto, a local high-frequency magnetic field B_{1f}′ that is perpendicular to the high-frequency magnetic field B_{1z} is produced in an area, shown in dashed lined, around the coil arrangement. This effect can be seen in FIG. 4, where the arrows respectively represent the direction and the strength of a magnetic field. In the coil S1 with its coil vector A running along the coil axis G, the high-frequency magnetic field B₁=B_{1z}·cos α is induced by the active high-frequency magnetic field B_{1z}, which high-frequency magnetic field in B₁=B_{1z}·cos α is amplified on account of the resonant circuit to give B₁′≈B_{1z}·=Q·B_{1z}·cos α. The projection of the induced high-frequency magnetic field B₁′ onto a straight line running perpendicular to the high-frequency magnetic field B_{1z} results in a component of the high-frequency magnetic field B_{1f}′ that runs perpendicular to the high-frequency magnetic field B_{1z}, with B_{1f}=B₁′·sin α≈Q·B_{1z}·cos α·sin α. It can be seen that B_{1f}′ is at a maximum for an angle of α=45°+n·90° with n=0, 1, 2, 3, . . . , and disappears altogether for α=m·90° with m=0, 1, 2, 3, . . . .

In the region shown in dashed line in FIG. 3, the high-frequency magnetic field B_{1f}′ excites a nuclear resonance signal which runs perpendicular to the main magnetic field B₀ and can thus be detected in a known manner by the high-frequency coil arrangement RF. Since in the rest of the examination area no nuclear resonance signal is excited, the high-frequency coil arrangement RF thus only detects the nuclear resonance signal from the vicinity of the coil arrangement having the coil S1 and the capacitance K1, since only here are there components of the high-frequency magnetic field B_{1z} which is being excited that are perpendicular to the main magnetic field B₀. As an alternative or in addition, this nuclear resonance signal in the vicinity of the coil arrangement can also be detected using the high-frequency coil arrangement z-RF if the latter is additionally used as a receiving unit (the corresponding coupling to the receiver RX is not shown in FIG. 2). This is possible since the excited nuclear resonance signal is coupled back in the high-frequency coil arrangement z-RF and is rotated in the z-direction in a manner analogous to the above-described mechanism (reciprocity of the inductive coupling).

If the object on which the coil arrangement shown in FIG. 3 is located can move, then it cannot always be ensured that the coil axis G of the coil S1 forms an angle of approximately 45° with respect to the direction of the high-frequency magnetic field B_{1z} or of the main magnetic field B₀. Therefore, an arrangement of three independent but identical resonant coil arrangements is proposed, the coil axes or coil vectors A of which in each case form an angle of 45° with respect to the other two coil axes. As a result, under the influence of the high-frequency magnetic field B_{1z}, three high-frequency magnetic fields B_{1f}′ are generated, which in each case run perpendicular to the direction of the high-frequency magnetic field B_{1z}. By simple vector addition of these three high-frequency magnetic fields B_{1f}′, the situation is achieved that, irrespective of the position of the arrangement in the examination area, there is always a component that is perpendicular to the high-frequency magnetic field B_{1z}. Only with a symmetric alignment of the three coil vectors A with respect to B₀ do the three perpendicular components B_{1f}′ cancel each other out. On the one hand, however, the probability that a moving object will assume precisely this position is very low. On the other hand, the contemplations in the present text are idealized, and in a more complex contemplation there is still always a residual field B_{1f}′. In a further example of an embodiment, it is proposed, in order to increase this residual field strength, to increase the strength of the high-frequency magnetic field B_{1z} if the alignment of the coil arrangements in this position is ascertained by a large drop in the detected nuclear resonance signal.

In step 85 shown in FIG. 1a, for the local resolution of the nuclear resonance signal excited by the high-frequency magnetic field B_{1f}′, the gradient fields GX, GY and/or GZ which accordingly have a gradient in the x-, y- and z-direction are activated, and under the influence thereof the excited nuclear resonance signal is detected in step 86. Steps 85 and 86 correspond to known methods of detecting nuclear resonance signals in the case of “normal” image detection, so that at this point no further details are given and reference should be made to the corresponding technical literature. FIG. 5a shows for one dimension the local profile of a projection of a detected signal S(ω) from a known system, as described for example in DE19844762. The signal increase at the point x₁, which is caused by the local high-frequency magnetic field in the vicinity of the coil arrangement, is superposed with the other nuclear resonance signals. In most cases, it can be seen that the excessive increase in the signal is difficult to distinguish from the other nuclear resonance signals. By contrast, FIG. 5b shows that, with a method according to the invention, the excessive increase in the signal at the point x₁ is present as a single signal, since the other nuclear resonance signals are not detected by the coil arrangement RF. Thus such an excessive increase in the signal can be evaluated in a manner that is considerably better. The detected nuclear resonance signal is evaluated in step 87.

As a possible result, in each case a projection from one dimension can be detected and a two- or three-dimensional positional image can be reconstructed therefrom, on which image the coil arrangement shown in FIG. 3 can be seen as an excessive increase in the signal against a uniform background (the directions of the projection can be selected at will; however, they are preferably perpendicular to one another). This method is also known as “rapid localization”. Such a positional image can be superposed with a “normal” MR image of the same subject slice or subject volume acquired immediately before or after said positional image, so that the position of the object in the overall image can be seen by an observer. As an alternative, initially those image or volume elements which with their image or volume values represent the excessive increase in the signal can be determined in the positional image. These image or volume elements can then be highlighted in a “normal” MR image by means of a particular coloring. It is also possible to indicate the geometrical distances of the determined position with respect to a reference point (e.g. the image zero point). These distances can be superimposed as numerical values in addition to a “normal” image or can be made visible by means of a graticule in a “normal” image.

Furthermore, it is possible, instead of just one projection, to determine in each case a complete set of projections and to reconstruct a high-value image with the same local resolution as in the case of the “normal” images. Since as the local resolution increases not only does the accuracy of the position increase but also the cost of reconstruction, as a compromise the local resolution can be reduced by subscanning, so that on the one hand more information than that of three projections is used in the reconstruction but on the other hand the cost of reconstruction is reduced compared with “normal” images.

In step 88, the question is asked whether the examination is finished. If not, the previously described steps starting from step 83 are repeated. Otherwise, the method is terminated.

Another example of embodiment is what is known as “tracking”, in which the intention is to follow the position of a moving object during an examination. As an example of this, in the text which follows a description is given of the following of the position of a catheter tip with automatic selection of the slices of the subject that are selected in the subsequent acquisition. In order to locate and navigate using nuclear magnetic resonance, three resonant coil arrangements 44, 46 and 48 are arranged at the tip of a catheter 40, the coil axes of which coil arrangements form an angle of approximately 45° with respect to one another. For this purpose, a first resonant coil arrangement in the form of a coil 44 is fitted to a cylindrical carrier 42, the windings of which coil are wound such that their coil axis D44 runs obliquely with respect to the carrier axis 42a. For the sake of clarity, the capacitor required to construct a resonant circuit is not shown in this case, nor in the case of the other coil arrangements. Furthermore, a second resonant coil arrangement in the form of a saddle coil 46 is fitted to the surface of the cylindrical carrier 42. Only the front areas of the conductors of the saddle coil 46 can be seen, and these extend on the rear side of the catheter 40 symmetric with respect to the front side. A third resonant coil arrangement likewise comprises a saddle coil 48 arranged on a cylindrical carrier, which saddle coil is constructed in the same manner as the saddle coil 46 but is arranged in a manner such that it has been rotated through approximately 45° with respect thereto. As a result, the coil axes D46 and D48 of the saddle coils 46 and 48 form an angle α1 of approximately 45°. The coil axis D44 of the first coil is aligned such that it forms both an angle α2 with the coil axis D46 and an angle α3 with the coil axis D48, each of these angles being approximately 45°.

If the catheter 40 with the shown coil arrangement is introduced into the body through blood vessels, the blood flowing through the vessels can be used as the imaging substance in the MR device. In order to fully use the perpendicular components of the high-frequency magnetic field that is being excited, which perpendicular components are generated by the coil arrangement, it is advantageous for the blood or an aqueous liquid to be present not only outside the carrier 42 but also within the interior. This can be achieved by means of openings in the carrier 42. As an alternative, it is possible to configure the carrier 42 in a closed manner, and to arrange a probe (not shown here) in the interior of the carrier 42. Such a probe consists of a material which, under the influence of corresponding magnetic fields, supplies nuclear resonance signals and is designed for example in the form of a water-filled capsule, it being possible for the water to be additionally mixed with a contrasting agent to increase the signal.

Using such a catheter 40, which is introduced into a subject under examination and is located in the examination area of the MR device shown in FIG. 2, the method shown in FIG. 1b is carried out, where the steps shown in said FIG. 1b correspond in principle to the steps shown in FIG. 1a. Initially, in step 82, the main magnetic field is switched on, then in step 84 a high-frequency magnetic field briefly becomes active on account of the high-frequency coil arrangement z-RF, and in step 85 the gradient magnetic field GZ, which in the z-direction runs parallel to the main magnetic field B₀, is activated. In step 86, the nuclear resonance signals excited in the vicinity of the catheter tip 40 are received, and these are evaluated in step 87. By using the abovementioned gradient field, it is possible to determine, from the detected signals, in a manner similar to that shown in FIG. 5b, the position of the catheter tip along the gradient, that is to say along the z-direction. In the following step 84a, nuclear resonance signals are then excited and detected in a conventional manner in that slice of the subject in which the position of the catheter tip has been found, and a corresponding slice image is reconstructed. The determination of the position of the catheter tip along the z-axis is then repeated and a next slice image of the slice in which the catheter is now found is generated. If the MR device is suitable for generating volume images, then using this method the shown volume can be selected such that the catheter tip is located for example in the center of the volume with respect to the z-axis. A combination with the above-described superposition methods is also conceivable.

In an MR device such as that shown, for example, in FIG. 2, it may occur, during practical conversion of the coil arrangement z-RF, that no complete parallelism of the high-frequency magnetic field B_{1z} with respect to the main magnetic field B₀ is achieved, particularly in the edge regions of the examination area. In the case of the vectorial contemplation, this means that the high-frequency magnetic field B_{1z} itself already contains components that are perpendicular to the main magnetic field B₀. These perpendicular components of the high-frequency magnetic field B_{1z}—even if they are only small—excite further nuclear magnetizations, the nuclear resonance signal of which is detected in addition to the nuclear resonance signal excited in the vicinity of the conversion means. In FIG. 5c, this is shown by way of example in the x-direction. In addition to the signal located at the position x1, further signals can be seen in the edge regions. As a rule, these signals are contained equally in various detections, since they stem from a more or less immobile subject under examination. If at the start of the examination a reference measurement is taken in which method steps 84 to 87 are carried out without any conversion means, then this reference measurement can be subtracted from the subsequent detected signals in order to eliminate these from the actually detected signals. If the conversion means are not located in the edge regions, then it is conceivable, as an alternative, to provide for band-pass filtering of the received signal in order to eliminate the edge region signals.

Finally, it should be pointed out that the methods described here are not restricted to catheter examinations. In general, the carrier 42 shown in FIG. 3 can also be arranged on other objects, for example on surgical instruments for minimally invasive operations or on markers for marking particular parts of the subject under examination (bones, organs, parts of the surface of the skin, etc.). When marking particular points on the surface of the subject, plasters may be used in which a miniaturized coil arrangement is incorporated. Furthermore, it is possible to provide a number of objects with conversion means, instead of just one object, and for the positions of these objects to be determined at the same time.

1. A method of determining the position of an object located in the examination area of an MR device, having the steps of
a) generating high-frequency magnetic field in the examination area, which high-frequency magnetic field runs essentially parallel to a main magnetic field that is active at the same time, whereby a component of the high-frequency magnetic field that is perpendicular to the main magnetic field being produced from the high-frequency magnetic field by conversion means fitted on the object, in the vicinity thereof,
b) detecting the nuclear resonance signal excited as a result of the perpendicular component of the high-frequency magnetic field, in conjunction with a gradient magnetic field,
c) evaluating the nuclear resonance signal and determining the position of the object.

2. An MR device for carrying out the method as claimed in claim 1, having
a) means for generating a main magnetic field in an examination area,
b) means for generating a high-frequency magnetic field in the examination area, which high-frequency magnetic field runs essentially parallel to the main magnetic field,
c) means for generating at least one gradient magnetic field,
d) means for detecting nuclear resonance signals,
e) an evaluation unit for evaluating the nuclear resonance signals,
f) a control unit for controlling the aforesaid components such that the following steps are carried out:
f1) generation of a high-frequency magnetic field in the examination area, which high-frequency magnetic field runs essentially parallel to a main magnetic field that is active at the same time, whereby a component of the magnetic high-frequency field that is perpendicular thereto being generated by conversion means fitted on the object, in the vicinity thereof,
f2) detection of the nuclear resonance signal excited as a result of the perpendicular component of the high-frequency magnetic field, in conjunction with a gradient magnetic field,
f3) evaluation of the nuclear resonance signal and determination of the position of the object.

3. A coil arrangement for an MR device for generating a high-frequency magnetic field in the examination area, which high-frequency magnetic field runs essentially parallel to the main magnetic field of the MR device.

4. Conversion means for an MR device for generating perpendicular components for a high-frequency magnetic field, having at least one coil arrangement with at least one coil, the coil axis of which forms an angle other than 90°, preferably an angle of 45°, with respect to the direction of the high-frequency magnetic field.

5. Conversion means as claimed in claim 4, wherein the coil arrangement forms a resonant circuit.

6. Conversion means for an MR device for generating perpendicular components for a high-frequency magnetic field, having at least two coil arrangements with in each case at least one coil, wherein the coil axes of the coils form an angle other than 90°, preferably an angle of 45°, with respect to one another.

7. A medical intervention instrument having an invasive portion that can be inserted into the body, on which portion conversion means for carrying out the method as claimed in claim 1 are arranged.

8. A catheter as claimed in claim 7.

9. A catheter as claimed in claim 8, having a carrier body that can be fitted to the catheter tip, on which carrier body three planar coil arrangements are fitted, wherein the coil axes of the coils in each case form an angle other than 90°, preferably an angle of 45°.

10. A computer program or computer program product, which enables the programmable components of an MR device to carry out a method as claimed in claim 1.
